(19) United States
(12) Patent Application Publication
(10) Pub. No.: US 2017/0273672 A1
(43) Pub. Date: Sep. 28, 2017

(54) BODY CAVITY SAMPLE COLLECTION DEVICE

(71) Applicants: Maarten Wiegerinck, Eindhoven (NL); Leo Raaijmakers, Leende (NL)

(72) Inventors: Maarten Wiegerinck, Eindhoven (NL); Leo Raaijmakers, Leende (NL)

(21) Appl. No.: 15/514,033

(22) PCT Filed: Sep. 25, 2015
(86) PCT No.: PCT/IB2015/002076

§ 371 Date: Mar. 24, 2017

Related U.S. Application Data
(60) Provisional application No. 62/055,744, filed on Sep. 26, 2014.

(51) Int. Cl. A61B 10/02; A61B 10/00

(52) U.S. Cl. CPC A61B 10/0283; A61B 10/0096; A61B 10/0291; A61B 2010/0074

(57) ABSTRACT

Inventive embodiments disclosed herein include a sampling device. The sampling device includes a tubular main body having a length and defining a lumen, the tubular main body defining a lumen that extends the length of the tubular main body, the tubular main body having a distal end sized for insertion into a body cavity and having a proximal end defining threads; and a flexible sample container, comprising threads for engagement with the tubular main body wherein the lumen of the tubular main body is in communication with the flexible sample container when the container is engaged.

### PRIORITY APPLICATION

This application claims the benefit of priority to U.S. Provisional Patent Application Ser. No. 62/055,744, filed Sep. 26, 2014, the content of which is incorporated herein by reference in its entirety.

### FIELD

Inventive subject matter disclosed herein relates to a body cavity sample collection device and to a method for collecting body cavity samples. For some embodiments, the body cavity is vaginal body cavity or an oral body cavity or a rectal body cavity.

### BACKGROUND

Obtaining a sample of tissue, cells and fluid compounds from a living being has been performed by many types of methods. Typically, the tissue, cell or fluid sample is transferred to a first vial at the site of the tissue extraction and one or more vials between the site of tissue, cell or fluid extraction and a site of testing.

### SUMMARY

One inventive embodiment includes a sampling device. The sampling device includes a tubular main body having a length and defining a lumen, that extends the length of the tubular main body, the tubular main body having a distal end sized for insertion into a body cavity and having a proximal end defining threads. The sampling device also includes a flexible sample container, comprising an attachment portion having threads for engagement with the tubular main body wherein the lumen of the tubular main body is in communication with the flexible sample container when the container is engaged with the tubular main body.

Another embodiment includes a sampling device that includes a tubular main body having a length and defining a lumen, that extends the length of the tubular main body, the tubular main body having a distal end sized for insertion into a body cavity and having a proximal end that includes a receiving portion defining threads, for receiving a flexible container.

Another embodiment includes sampling device that includes a tubular main body having a length and defining a lumen that extends the length of the tubular main body, the tubular main body having a distal end sized for insertion into a body cavity and having a proximal end defining an attacher-detacher mechanism.

One other embodiment includes a method of obtaining a tissue cell sample from a body cavity. The method includes attaching a flexible sample container to a tubular main body, the tubular main body having a distal end sized for insertion into the body cavity and having a proximal end defining an attacher-detacher, wherein the flexible sample container is attached at the attacher-detacher; introducing the distal end of the tubular main body into the body cavity; squeezing the flexible sample container; so that the prefilled fluid from the flexible sampler container is expelled into the body cavity ans mixes with cells, releasing the pressure on the flexible sample container so that a tissue cell sample is removed from the body cavity by vacuum force and transferred to the sample container; detaching the sample container from the tubular main body; and attaching a cap to the flexible sample container.

Another embodiment includes a sampling device. The sampling device includes a tubular main body having a length and defining a lumen, the tubular main body defining a lumen that extends the length of the tubular main body and defines a single opening centrally at a distal end of the tubular main body, the distal end sized for insertion into a body cavity and the tubular main body having a proximal end defining an attacher-detacher.

### DESCRIPTION OF DRAWINGS

FIG. 1 is a longitudinal cross-sectional view of one embodiment of a vaginal sample collection device of the present invention.

FIG. 2 is a longitudinal side view of the embodiment of FIG. 1 of a vaginal sample collection device.

FIG. 3 is a cross-sectional distal view of the embodiment of FIG. 1 of the vaginal sample collection device.

FIG. 4 is a cross-sectional proximal view of the embodiment of FIG. 1 of the vaginal sample collection device.

FIG. 5 is a perspective view of a collection container component of the vaginal sample collection device of FIG. 1.

FIG. 6 is a perspective view of another collection container component of the vaginal sample collection device of FIG. 1.

FIG. 7A is a cross-sectional front view of the distal end of a vaginal sample collection container component of the present invention showing a one-way valve.

FIG. 7B is a longitudinal side cross-sectional view of the distal end of the vaginal sample collection device of the present invention, showing a side view of the one-way valve of FIG. 7A.

FIG. 8 is a longitudinal cross-sectional view of another embodiment of a vaginal sample collection device of the present invention

FIG. 9 is longitudinal cross-sectional view of another embodiment of a vaginal sample collection device of the present invention.

FIG. 10 is a longitudinal side view of the embodiment of FIG. 9.

FIG. 11 is a side cross-sectional view of a collection container embodiment of the present invention.

FIG. 11A is a distal view of a collection container embodiment of FIG. 11.

FIG. 11B is a close up view of the threads of the collection container embodiment of FIG. 11.

FIG. 11C is a radial cross-sectional view of the collection container embodiment of FIG. 11.

### DETAILED DESCRIPTION

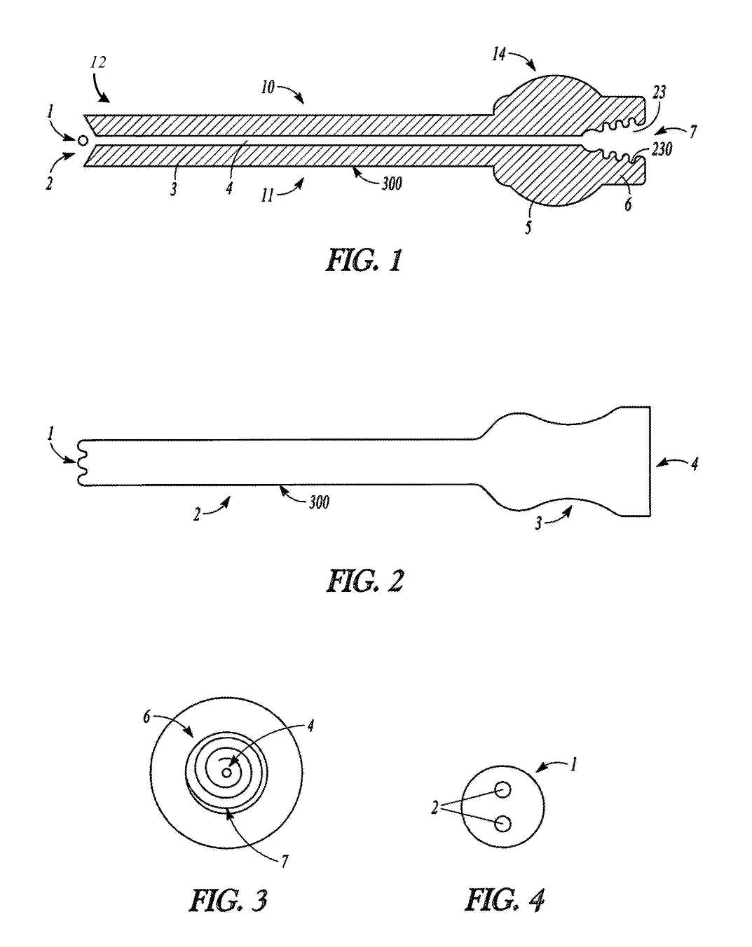

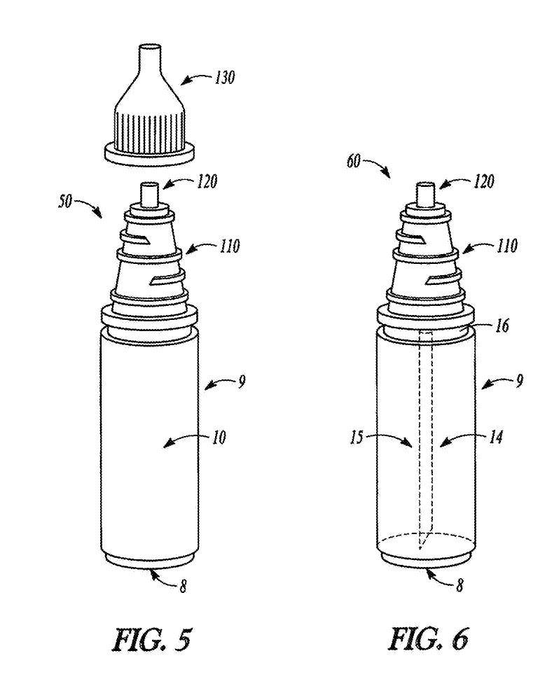

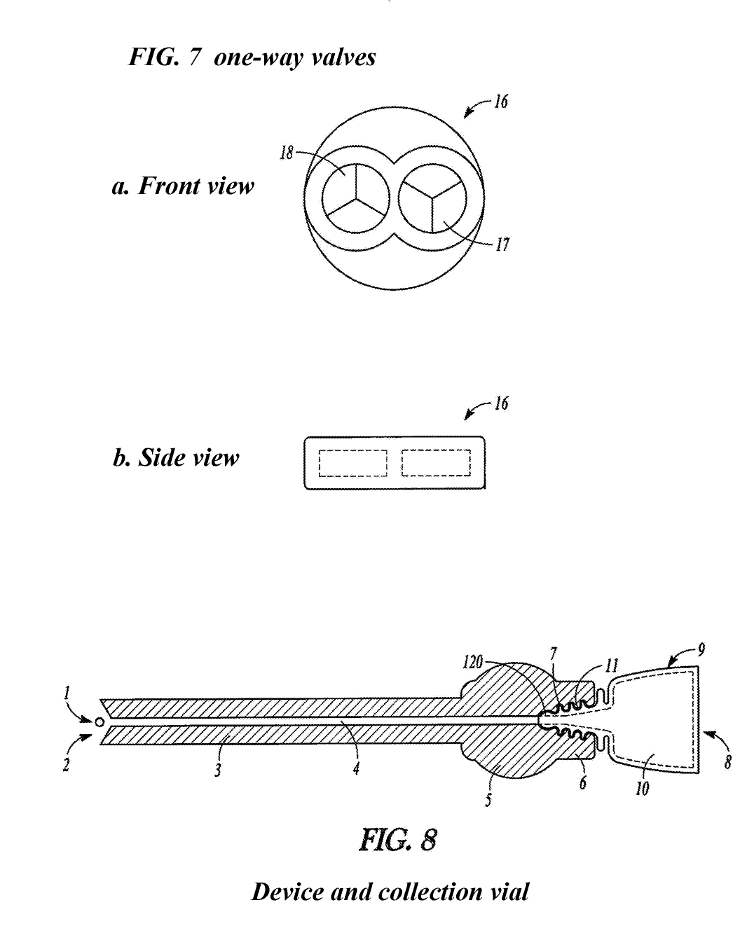

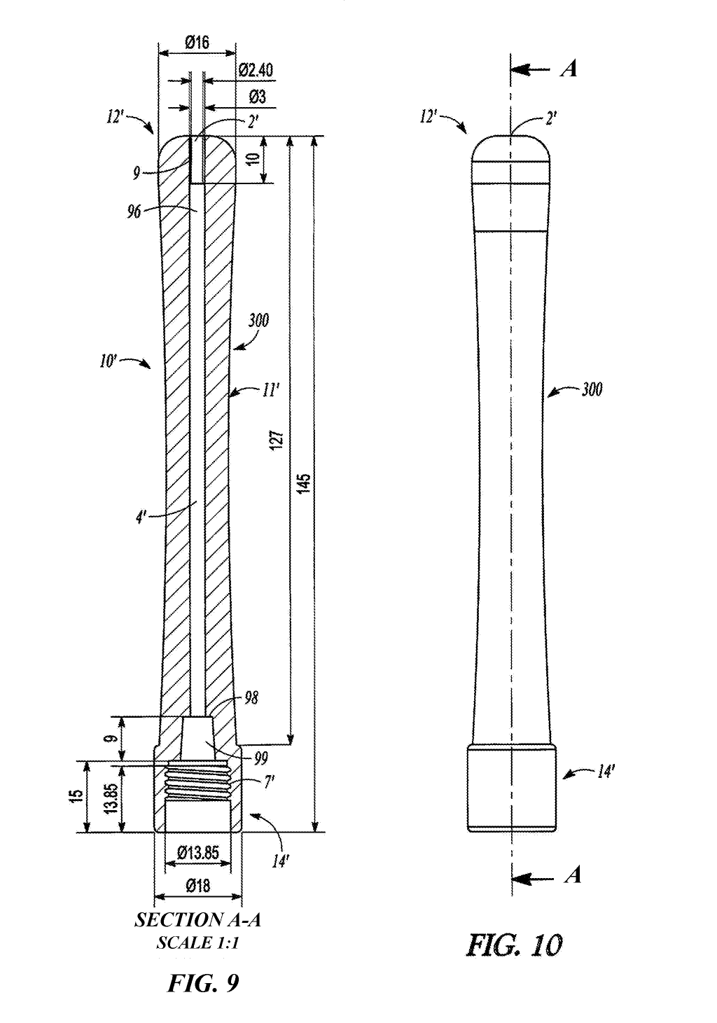

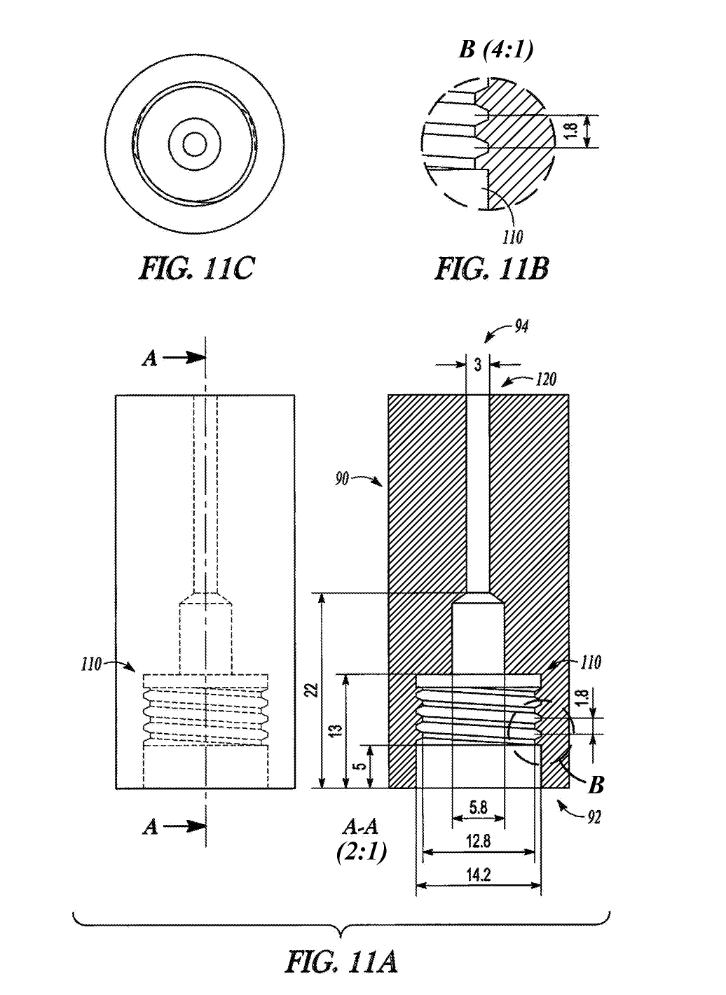

One inventive embodiment of a body cavity sample collection device is a vaginal sample collection device that is shown generally in longitudinal cross-section at 10 in FIGS. 1 and 10′ in FIG. 9. The device embodiments 10 and 10′ each includes an insertion part which includes a tubular main body 11 and 11′, respectively, defining a lumen 4 and 4′, respectively, each tubular main body 11 and 11′ having a distal end 12 and 12′ defining an opening, shown at 2 in FIGS. 1 and 4, and 2′ shown in FIG. 9. The insertion parts 10 and 10′are each insertable into a vaginal cavity of a patient and each of embodiments 10 and 10′, having a proximal end 14 and 14′, each having a threaded connection portion 7 and 7′, respectively, for receiving a flexible sample container. The vaginal sample collection device embodiments 10 and 10′ also each include a collection container, such as is shown at 50 in FIG. 5, 60 in FIG. 6 and 90 in FIG. 11 that is attachable to either of the main body 11 or 11′ at 7 in FIG. 1 and 7′ in FIG. 9, respectively.

The vaginal sampling device 10′ includes a single opening 2′ with a slight indent bordering the opening, shown at 9 in FIG. 9. The openings 2, shown in FIG. 1, and 2′, shown in FIG. 9, have a diameter within a single millimeter range.

It has been found that vaginal sampling devices having only side openings or an opening at the end 12 of the device with side openings leaked saline fluid from the vagina after flushing with saline solution, an unacceptable feature for patients. The end opening 2′ having a slight indent 9 of the vaginal sampling device embodiment of FIG. 9 forms an optimal area for recollection of a fluid sample and decreases the possibility of vacuum fixation of the top of the vaginal wall at recollection.

While a vaginal body cavity is described herein, it is understood that other body cavities such as an oral body cavity, or a rectal body cavity are accessible by the sample collection device embodiments that are sized for particular body cavity access.

For some embodiments, including the vaginal sampling device embodiments, the sample collection device embodiments 10 and 10′ include a liquid, such as a saline solution, for introduction into the vaginal body cavity. The saline solution acts as a transport medium for transporting a cellular tissue sample from the vaginal body cavity to the flexible sample container.

With the sample collection device embodiments 10 and 10′, a tissue sample from a vaginal body cavity or other body cavity is retrievable within a single fluid sample container 50, shown in FIG. 5, or 60 shown in FIG. 6 or 90, shown in FIG. 11. Each of the sample containers 50, illustrated in FIG. 5, 60 illustrated in FIG. 6, or 90, illustrated in FIG. 11 is usable for storing fluid for flushing the body cavity as well as transporting a tissue sample using a highly hygienic procedure for a patient and for lab workers analyzing the sample.

For the embodiment shown at 10′ in FIG. 9, the distal end 12′ of the device has a larger diameter than the diameter midway from the distal end 12′ at 300 and proximal end 14. For one embodiment, the distal end, 12′ diameter is 16 mm and the midway diameter at 300 is 13 mm. The wider distal end, compared to the lumen diameter, resulted in very little to no leakage. With the embodiment shown at 10′, the vaginal wall of a patient encloses the rounded distal end 12′ and prevents leaking. It is believed that the distal end 12′ of the embodiment 10′ is a bio mimic, in that its distal end shape is in the form of a penis. In one embodiment, the length of the vaginal sampling device embodiment 10′ is 100 mm. These dimensions impart optimal comfort to the patient and function.

For one embodiment, the diameter of the lumen 4′ is 2.4 mm at the tip of the tubular main body and 3 mm at the distal end 2′ proximal to the indent 9′. These dimensions facilitate a formation of a spray while expelling fluid into the deepest part of the vagina, which mobilizes cells from the cervix and vaginal pouch of a patient, for sample collection. The 3 mm lumen allows a fluent sample recollection fluid stream into a sample container. While specific dimensions are disclosed herein, it is understood that other dimensions effective for forming a spray for cellular sample collection are usable.

In one embodiment, the device 10′ main body includes shallow indentations to facilitate the handling of connecting the proximal section 14′ of the main body to the bottle 50 in FIG. 5, or 60 in FIG. 6, or 90 in FIG. 11, the insertion of the distal end 12′ into the vagina of a patient, and the removal of the bottle after sample collection.

At the proximal end 98 of the lumen 4′, through an inserter shaft 99, there is a portion where the tip of a sampling container as shown in FIG. 11 at 94, fits seamlessly, in such a way that the opening of the lumen at the proximal inserter side matches the opening of the tip of the sampling container 98 shown in FIG. 9, forming a continuum when both parts are connected.

One sample container embodiment, illustrated at 90 in FIG. 11, includes a bottle 92 with a tip 94. The tip 94 is sized and shaped to fit on and within the proximal inserter portion 23 or 99 of the sampling device 10 or 10′, respectively. The tip 94 is straight and not tapered.

The single fluid sample container 50 or 60 or 90 is opaque or translucent. For some embodiments, users can see that a sample has returned to the bottle 50, 60 or 90. For some embodiments, 3 to 3.5 ml of saline is optimal for collection of a high DNA sample yield without patient discomfort. More than 3.5 ml of saline resulted in fluid leakage for the vaginal sampling device embodiment. The sample container is, for some embodiments, a 5 ml bottle.

For one embodiment, illustrated generally at 60 in FIG. 6, the collection container 60 includes two separate compartments 14 and 15 with two one-way valve systems, positioned at 16, for outflow of sterile non-toxic saline solution and inflow of a sample into preservation fluid in the collection container. The one-way valves are shown at 17 and 18, respectively, in FIG. 7A, near the neck of the container. One compartment is connected to a one-way out stream valve and the other compartment is connected to a one-way in a stream valve. The compartment with the out stream valve contains 5-10 ml sterile saline solution, 0.9% sodium chloride. The compartment with the in stream valve contains 3-5 ml preservation fluid. This container embodiment renders superfluous, a step of transferring a sample in a separate container with preservation fluid that is typical of other sampling methods. This additional step harbors risk for the user because of the toxic content of the preservation fluid or the infectious content of the sample.

Another sample container embodiment, illustrated at 50 in FIG. 5, 60 in FIG. 6 and 90 in FIG. 11, includes a sample container having a flexible medical grade main body. Typically, the sample containers 50, 60, and 90 are made of a flexible polymeric material. The sample containers 50, 60 and 90 have a flexibility that enables them to be squeezed.

The connection for the separate sample container 50 or 60 or 90 to the tubular main body 11 or 11′ enables sample collection in the following exemplary embodiment: The distal tubular main body 11 or 11′ is introduced into the vagina of a patient. The sample container 50 or 60 or 90 includes a lavage fluid, such as a saline solution, that is introduced into the vagina by squeezing the sample container so that the lavage fluid is transported through the lumen of the tubular main body into the vagina. A vaginal fluid sample is retrieved by releasing pressure from squeezing the sample container. The sample container is disconnected from the tubular main body. The sample container is closed with a screw cap and transferred to a laboratory for analysis of the sample.

While threads are shown for attaching the flexible sample container to the tubular main body, it is understood that other types of attaching/detaching mechanisms are suitable. The other types include magnets and bayonet type attaching mechanisms

The sample collection device embodiments of the present invention do not require a transfer of a sample from a sampling device into a separate sample transport container. Instead, the sampling container receives the sample while it is attached to the tubular main body. Thus, risks of contaminating the exterior part of the container with biological material of the sample, as well as spillage of the sample or loss of the sample by accidently dropping of the container are eliminated. The present inventive embodiments are hygienic to a user, because the user has no contact with the sample. No separate container is required for lavage and sample transport. Sample identification is reliable because the sample container is labelled before it is detached from the longitudinal sampler. The sample is directly collected into the sample transport container. The sample collection device embodiments eliminate mistakes of putting the wrong identification on a separate container. The handling procedure for the user is easy, time saving and sample taking is reliable. When at a laboratory, the sample is removed through the opening at the top of the sample container with a pipette. The handling is hygienic during transport. There is no contamination of the sample container with the biological sample.

The lavage method described herein produces a higher DNA yield of vaginal and cervical substances. Lavage is atraumatic with no tissue damage. No separate transport medium is needed. The lavage method, as used herein, is the method of introducing liquid such as a saline solution into a body cavity, such as a vaginal body cavity.

For the sampling device of 10 in FIG. 1, the tubular main body 11 of a vaginal body cavity collection device has a diameter of approximately 1 cm and a length of approximately 10 cm and ends at the proximal end 7 in a grip part 14 and a distal body with an opening 23 at the back side. The total length of the device is approximately 14 cm. The insertion part 1 has a rounded top with one or more openings connected to a small lumen at the central inside of the tubular body reaching from the proximal opening 2 to the top of the device. The opening 23 at the back of the device has a spirally tapered form towards the lumen.

The sample container 50 or 60 or 90 is, for some embodiments, a flexible medical grade container with a small open tip 120 and a tapered spiral screw neck 110 together with a screw cap 130. For some embodiments, the sample container 50 or 60 or 90 includes five to ten mls of a sterile saline solution, i.e. 0.9% by weight sodium chloride. The sample container includes three components, a bottle component, a tip at the opening of the bottle and a screw cap. The tip acts to reduce the force of the fluid rendering it more controllable. The tip also reduces steps for fluid testing.

One procedure embodiment for using the sampling device embodiments of the present invention includes the following steps for a user:
Remove the tubular main body from packaging;
Remove the sample container from packaging;
Assemble the sampling device by screwing the sample container to the back opening of the tubular main body;
Grip the assembled sampling device and introduce the rounded top of the tubular main body into the body cavity. In the case of vaginal wall sampling, the insertion is approximately 7 cm, which is, for some embodiments, marked on the tubular main body;
Hold the sample container between thumb and fingers and squeeze until the saline solution is expelled into the body cavity;
By releasing the pressure on the sample container, the expelled fluid mixed with body cavity content comprise the sample, which flows back into the sample container;
Disassembling the tubular main body from the sample container by unscrewing the two parts;
Closing the sample container with the screw cap and transport to a laboratory for analysis of the sample;
At the laboratory, the screw cap is removed and a pipette is inserted in the container tip opening to collect the sample;

For the embodiment wherein the sample container includes compartments, the container is squeezed so that saline solution is expelled through the one-way out stream valve into the body cavity while the in stream valve stays closed. By releasing the pressure the fluid sample flows back into the container compartment with the preservation fluid through the one way in stream valve;
Immediate collection into preservation fluid of a sample is required in case of detection of mRNA or unstable proteins.

For some embodiments, the sample collection device is usable for sampling the vaginal cavity for detection of genital bacteria, viruses, protozoa, fungi which can affect health or cause discomfort comprising sexually transmitted infection species and protective bacillus. The vaginal cavity is, for some embodiments, sampled for detection for HPV genotypes and molecular markers related to the development of cervical cancer. The sampling device is usable for sampling for vaginal and cervical species for detection of bacteria and virus in pregnant women which are related to early birth, intra-uterine fetal death and neonatal morbidity and mortality. The sample collection device is usable for sampling the oral cavity for detection of bacteria, viruses and fungi related to infections of the oral-pharyngeal cavity and respiratory tract and oral-pharyngeal cancer. The sampler is usable for sampling of the rectal cavity for detection of bacteria, virus, protozoa and fungi related to gastrointestinal infections and diseases.

Sampling device embodiments disclosed herein pose no risk of sample contamination. The sample is collected from the vagina into the sampling container. There is no contact by the collector with the sample. There is no transfer of fluid collected from the vagina. At the lab, there is no handling contact of a technician with the sample, reducing handling by the lab. A technician shakes the sampling container, removes the cap, and drops the sample on an analysis wet plate. Transfer requires no pipetting and no change of gloves. This simplicity is a significant improvement over conventional sampling which requires a technician to change gloves, remove a brush tip, add the brush tip into a fluid container, and apply the sample to a plate.

For some embodiments the sampling container attaches to the sampling device 10 or 10′ by threads such as are shown at 110 in FIGS. 11, 11A, 11B and 230 in FIG. 1. Other embodiments include a bayonet mount. For some embodiments, the sampling device and sampling container include vertical indicators that are aligned when the sampling container is securely attached to the sampling device. For one embodiment, the sample container includes a horizontal indicator for determining the amount of sample fluid collected.

The sample collection device also has veterinary applications. These applications include sampling of the vaginal cavity of horses to detect bacteria and viruses related to recurrent miscarriage and still birth. Sampling of the oral cavity of horses and cattle is performed to detect bacteria and viruses related to respiratory infections and diseases. Sampling of body cavities of other primates is performed for detection of infectious diseases.

In various embodiments, the inventive composition or method can be any one of any of the combinations and/or sub-combinations of the above-listed embodiments.

While the invention has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference to the same extent as if each individual publication was specifically and individually indicated to be incorporated by reference.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A sampling device comprising:
a tubular main body having a length and defining a lumen, wherein the lumen extends the length of the tubular main body, the tubular main body having a distal end sized for insertion into a body cavity and having a proximal end defining threads; and
a flexible sample container, comprising threads for engagement with the threads of the proximal end of the tubular main body wherein the lumen of the tubular main body is in communication with the flexible sample container when the container is engaged.

2. The sampling device of claim 1, wherein the flexible sample container includes a screw cap.

3. The sampling device of claim 1, wherein the flexible sample container encloses a saline solution.

4. The sampling device of claim 1, wherein the flexible sample container encloses a preservative.

5. The sampling device of claim 1, wherein the flexible sample container defines two compartments within the container.

6. The sampling device of claim 1, wherein the lumen has a diameter that is smaller at the distal end of the tubular main body than midway the length of the tubular main body.

7. The sampling device of claim 1, wherein the distal end of the tubular main body has a diameter greater than the diameter midway the length of the tubular main body.

8. A sampling device comprising:
a tubular main body having a length and defining a lumen, wherein the lumen extends the length of the tubular main body, the tubular main body having a distal end sized for insertion into a body cavity and having a proximal end defining an attacher.

9. The sampling device of claim 8, wherein the lumen has a diameter that is smaller at the distal end of the tubular main body than midway the length of the tubular main body.

10. The sampling device of claim 8, wherein the distal end of the tubular main body has a diameter greater than the diameter midway the length of the tubular main body.

11. The sampling device of claim 8, wherein the attacher comprises threads.

12. A method of obtaining a tissue cell sample from a body cavity, comprising:
attaching a flexible sample container to a tubular main body, the tubular main body having a distal end sized for insertion into the body cavity and having a proximal end defining an attacher-detacher, wherein the flexible sample container is attached at the attacher-detacher;
introducing the distal end of the tubular main body into the body cavity;
squeezing the flexible sample container;
releasing the flexible sample container so that a tissue cell sample is removed from the body cavity and transferred to the sample container;
detaching the sample container from the tubular main body;
attaching a cap to the flexible sample container.

13. The method of claim 12, wherein the flexible sample container includes a liquid that is transferred to the body cavity when the flexible sample container is squeezed and is transferred, with tissue cells, to the flexible sample container, when the flexible sample container is released.

14. The method of claim 12, for sampling the vaginal cavity of an organism to detect bacteria, viruses and protozoa.

15. The method of claim 12, for sampling the oral cavity of an organism to detect bacteria, viruses and protozoa.

16. A sampling device comprising:
a tubular main body having a length and defining a lumen, the tubular main body defining a lumen that extends the length of the tubular main body and defines a single opening centrally at a distal end of the tubular main body, the distal end sized for insertion into a body cavity and the tubular main body having a proximal end defining an attacher-detacher.

17. The sampling device of claim 16, wherein the distal end of the tubular main body defines an indentation bordering the single opening of the lumen.

18. The sampling device of claim 16, wherein the distal end has a diameter of 16 mm and the lumen has a diameter of 13 mm and the sampling device is used to sample a vagina.

19. The sampling device of claim 16, wherein the sampling device has a length of 100 mm.

20. The sampling device of claim 16, wherein the diameter of the lumen is decreased to 2.4 mm at the distal end.

21. The sampling device of claim 16, further including a sampling fluid.

22. The sampling device of claim 12, wherein in the tubular main body defines indentations.

23. The sampling device of claim 16, wherein the lumen has a diameter that is smaller at the distal end of the tubular main body than midway the length of the tubular main body.

24. The sampling device of claim 16, wherein the distal end of the tubular main body has a diameter greater than the diameter midway the length of the tubular main body.
